(19) United States
(12) Patent Application Publication
(10) Pub. No.: US 2002/0193882 A1
(43) Pub. Date: Dec. 19, 2002

(54) Trial balls for hip joint prostheses

(76) Inventor: Hansjorg Koller, Winterthur (CH)

Correspondence Address: CHRISTIE, PARKER & HALE, LLP, 350 WEST COLORADO BOULEVARD, SUITE 500, PASADENA, CA, 91105, US

(21) Appl. No.: 10/141,479

(22) Filed: May 8, 2002

(30) Foreign Application Priority Data
May 18, 2001 (EP) 01810500.7

(51) Int. Cl. A61F 2/36

(52) U.S. Cl. 623/23.12; 623/23.14

(57) ABSTRACT

The invention deals with trial balls for hip joint prostheses which have a cone connection between a prosthesis neck (24) with a cone (3) and fittable hip joint balls, with the hip joint balls and the trial balls (1) each having practically the same radii (21) and each having the same position of their conical counter surfaces (4) relative to the ball center (11). The trial balls have a separate insert (2) with a pre-determined spring effect for the contact to the cone (3), with the insert being able to be secured in the trial balls (1) at a different spacing (27) along the polar axis (13) to the ball center (11).

The invention deals with trial balls for hip joint prostheses which have a cone connection between a prosthesis neck with a cone and fittable hip joint balls, with the hip joint balls and the associated trial balls each having practically the same radii and each having the same position of their conical counter surfaces relative to the centre of the ball.

A widespread surgical technique with artificial hip joints includes first providing trial balls, which correspond in the position of their conical counter surfaces relative to the ball centre to the artificial hip joint balls, after the insertion of a prosthesis shaft with a cone connection for an artificial hip joint ball, in order to check the tension of the ligaments in the joint on articulation. The trial ball can have a slightly smaller diameter, for example a reduction by 0.3 mm, so that it can be inserted more easily into a bearing shell of the acetabulum. The physician can in this way find the ball with the optimum penetration depth for the cone with balls which can be easily removed from the shaft and which are as a rule made of plastic, so as not to damage the ball shell, and replace said ball with the final hip joint ball which is anchored on the cone with a fixed force fit.

An analogue surgical technique provides setting the trial balls on a driven in bone rasp, which has the prosthesis shape and a same cone, in order to check the articulation. The cone of the rasp then frequently has an additional groove or a cut-out in order there to transmit the forces onto the rasp on the driving out of the rasp.

A known measure consists of applying a cut into the conical counter surface and applying an O ring there which protrudes over the conical surface. This O ring should maintain its form despite multiple pressing and sterilisation.

After the trial balls are set on, it therefore frequently occurs that the conical surfaces separate from one another - for example by brushing against another object. The invention is intended to counter this. It has the object of preventing an unintentional sliding of the trial balls off the cone. This object is met in that the trial balls have a separate insert with a predetermined spring effect for the contact to the cone, with the insert being able to be fastened in the trial balls at a different spacing along the polar axis to the ball centre.

The invention has the advantage that the holding force for a trial ball is not influenced by the usual dimension deviations such as manufacturing tolerances and long term use. The holding force remains practically unchanged even with a displacement by tenths of millimeters in the longitudinal axis.

A further advantage consists of the fact that the trial balls can be made of a rather soft plastic, while the insert can be made of a material with a high load capability.

The dependent claims 2 to 12 represent advantageous further developments of the invention.

The invention has the further advantage that a standard insert can be used for one cone size which can be inserted in trial balls of different ball diameter and in trial balls of the same ball diameter and of a different position of the conical counter surface to the ball centre.

The insert can be manufactured from an injection mouldable plastic in an injection moulding tool in order to obtain the same dimensions over a large number. However, it can also be made of metal bars on a bar automatic lathe due to the accessibility for working tools.

If the insert has the shape of a beaker whose rim is divided into supporting tongues and flexion springs by longitudinal slots and if the flexion springs are provided with inside cams projecting over the conical counter shape, the cone is guided by the support tongues on insertion and pressed radially by the inside cams of the flexion springs. Under the influence of these radial pressing forces, the friction at the inside cams prevents an accidental sliding off of the cone. The support tongues can additionally be radially supported in the trial ball in order to prevent over-straining in a tilted insertion of the cone.

If longitudinal grooves are applied at the inner side of the trial ball, these can be used with appropriate outer cams of the flexion springs as security against turning for the insert. However, they can also serve as a mount in the clamping of the semi-finished trial ball for the manufacture of the ball surface. Both parts can be put together by a catch connection in which the insert is pressed lightly together and the trial ball is slightly expanded. The position of the inner cams at the insert can be provided in the longitudinal direction such that grooves or recesses at the cones are also engaged behind by the cams at the cones of rasps for the same cone size in order to prevent an unwanted sliding off.

In the cones of the shaft prostheses, the surface is frequently provided with a very fine thread whose peaks can deform slightly in order to prevent local tension peaks with hard prosthesis balls of metal or of ceramics. Such very fine threads simultaneously increase the friction between the inner cams and the cone and prevent the cone from sliding off. The wear at the inner cones is kept small by the radial forces of the flexion springs being controllably pre-determined. Unlike a trial ball with a conical force fit, the connection to the conical counter surface cannot be overloaded since the deep abutment for the cone is provided at its end face.

The invention is described in the following by way of embodiments. There are shown:

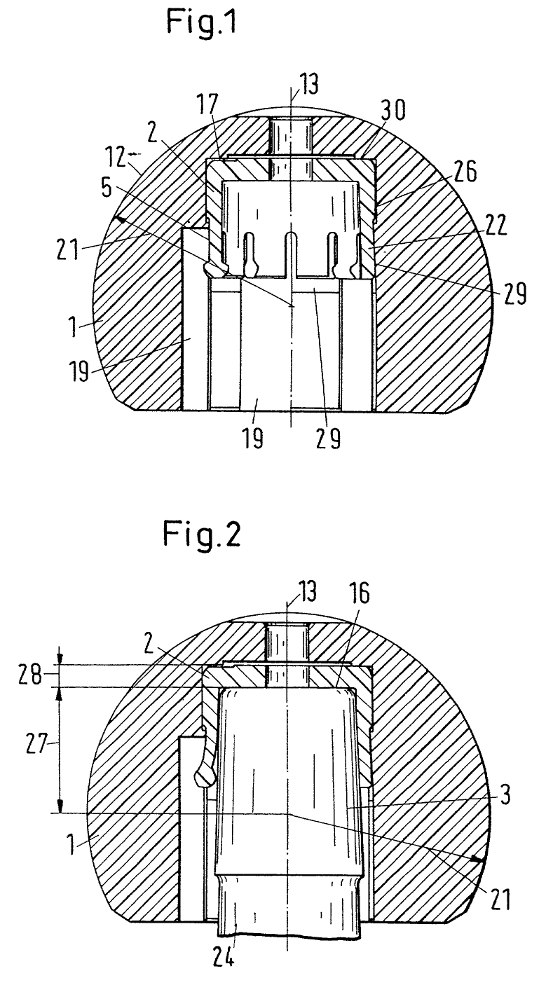

FIG. 1 schematically, an enlarged longitudinal section through a trial ball of the invention;

FIG. 2 schematically, an enlarged longitudinal section through the trial ball of FIG. 1 which is mounted on the cone of a shaft prosthesis;

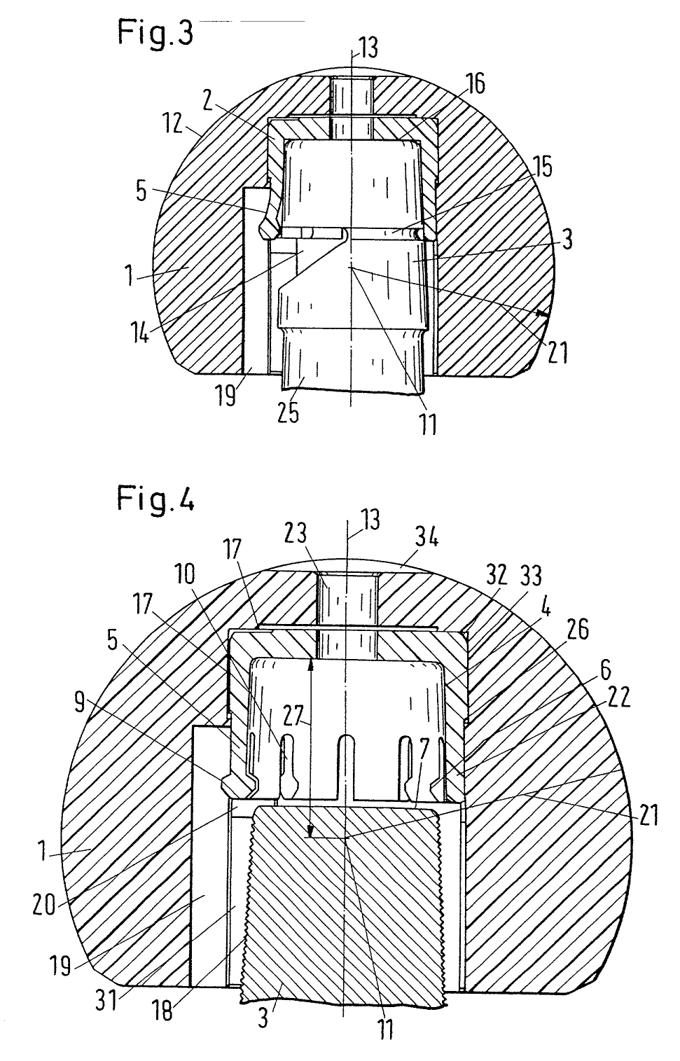

FIG. 3 schematically, an enlarged longitudinal section through the trial ball of FIG. 1, which is mounted on the cone of a rasp;

FIG. 4 schematically and in a great enlargement, a longitudinal section through a trial ball of FIG. 2 on mounting onto a cone;

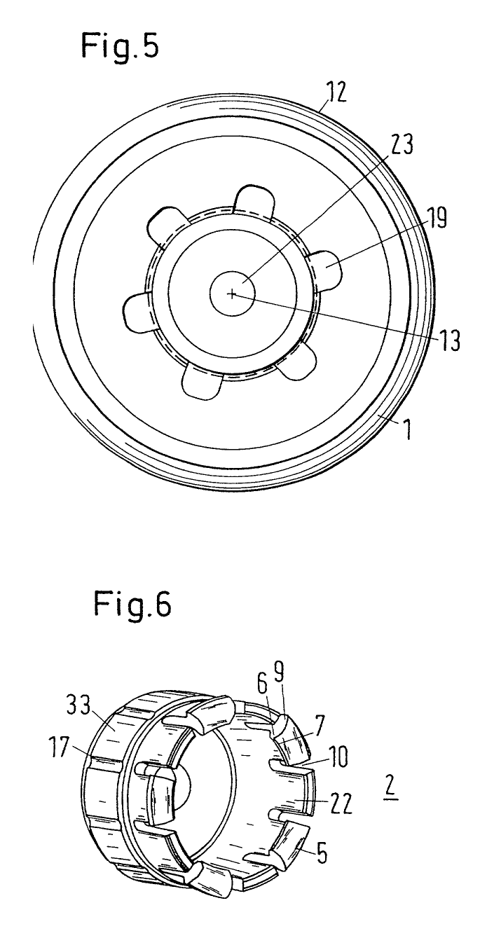

FIG. 5 schematically and enlarged, a view from below of the trial ball of FIG. 12, in which the insert has not yet been mounted;

FIG. 6 schematically and enlarged, a general view of the insert of FIG. 1 before its securing in the trial ball; and

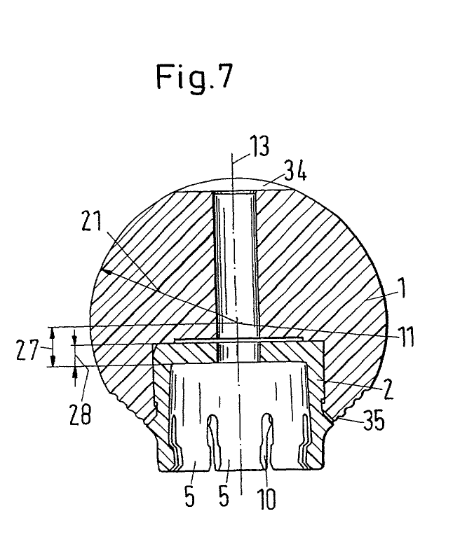

FIG. 7 schematically and enlarged, a trial ball with an insert projecting out of the trial ball.

The Figures show trial balls for hip joint prostheses which have a cone connection between a prosthesis neck 24 with a cone 3 and fittable hip joint balls, with the hip joint balls and the trial balls 1 each having practically the same radii 21 and each having the same position of their conical counter surface 4 to the ball centre 11. The trial balls have a separate insert 2 with a pre-determined spring force for the contact to the cone 3, with the insert 2 being able to be fastened in the trial balls 1 at a different spacing 27 along the polar axis 13 to the ball centre 11.

The same reference numerals are used for the same functions in the Figures.

The trial ball in FIGS. 1, 2, 3, 4 and 5 consists of an actual ball body 1 made of plastic, for example of POM (polyoximethylene), which has good machining properties and does not injure other objects. Still more than half of the ball surface 12 is present. The ball is drilled open from below along its polar axis 13 and an insert 2 is inserted into this bore and held by a catch connection 26. The end face 30 of the insert has contact in the ball 1 and has a conical transition to a radially projecting cylindrical projection 33 which is caught in a recess of the bore. The insert 2 itself has the shape of a beaker with a conical counter surface 4 which is intended for the mounting of a cone 3 and which is dimensioned such that the end surface of the cone 3 abuts the beaker base prior to the widening of the conical counter surface 4. The conical counter surface 4 thus has only one function as a guide and a radial support. Longitudinal slots 10 are made up to half the depth of the beaker and each separate support tongues 22 and flexion springs 5 in a peripheral direction. The ball 1 is worked on the inner side in accordance with the division into support tongues 22 and flexion springs 5. Conical part surfaces 29 are applied in the ball for the support tongues 22 which extend the conical counter surface 4 to the beaker edge in order to give the cone 3 a longer guiding. The conical part surfaces 29 in turn support the support tongues 22 as support areas 20 when a cone 3 is inserted in a tilted manner. The production of the conical parts surfaces 29 is achieved by a conical surface first being bored in the bore, said conical surface merging into a cylinder 31 toward the base.

Subsequently, radial elongate grooves 19 are applied in a width such that the flexion springs 5 can spring back into these grooves in a radial direction. The flexion springs 5 have inner cams 6 and outer cams 9 towards the beaker rim. The outer cams 9 project outwardly so much that they lie in the elongate grooves 19 even in the unloaded state of the flexion springs 5 and form a security against turning 8 for the insert 2.

The actual holding force is determined by the projection of the inner cams and the strength of the flexion springs which may only be loaded in their elastic region. When a cone is inserted, the flexion spring must spring back by the dimension of the projection of the inner cam 6 into the elongate grooves 19. A run in surface 7 ensures that the inner cam 6 is slowly pressed outwardly. A pressing force onto the cone 3 results in accordance with the spring path of the flexion spring 5 which is pre-determined and which produces so much friction between the inner cams 6 and the cone 3 that sliding off the cone 3 is prevented. With a symmetrical distribution of the flexion springs 5, the pressing forces at the cone 3 largely cancel one another out and have a centring effect, while the friction forces produced by the pressing forces are summed to prevent sliding off the cone 3. A holding force produced in this way cannot be used only with trial balls with a cone, but also with trial balls having a cylindrical seat with respect to their shafts.

Star-shaped passages are worked into the end surface 30 of the insert 2 which allow ventilation up into the actual catch connection 26 and give security during sterilisation. The insert 2 and the trial ball 1 have a bore 23 in the polar region through which the end face 16 of an inserted cone 3 can be controlled. The pole of the trial ball 1 can have a smoothed surface or recess 34 in which an inscription has been applied for its identification.

A cone 3 is shown in FIG. 4 with the previously mentioned very fine thread 18. The spacing 27 between the beaker base and the centre 11 of the trial ball 1 is selected in accordance with the provided spacing 27 between the centre of a femur ball and the end surface of the fastened cone 3 in order to anchor the insert 2 in the trial ball 1. The spacing 27 must be enlarged by one working dimension 28 for the deep abutment of the insert 2.

In FIG. 3, the neck 15 of a rasp (not shown) merges into a cone 3 onto which a trial ball 1 with an insert 2 can be mounted in order to check the articulation with a rasp driven into the femur bone. The cone 3 of the rasp is provided with a cut-out 14 which serves for the transmission of force on the driving out of the rasp. The cone 3 is furthermore provided with a channel 15 into which the inner cams of the flexion springs 5 latch when the end face 16 of the cone 3 abuts the base of the insert 2.

The insert 2 in FIG. 6 is made of injection moulded plastic from the group of polyether ether ketones (PEK, PEEK, PAEK, PEEKK, etc.) and is provided for trial balls 1 of different radius 21 and a different spacing 27 from the ball centre 11 to the end face 16 of a same cone 3. The elongate slots 10 can be seen which divide the beaker-shaped insert 2 at its rim into support tongues 22 and into flexion springs 5 with inner cams 6 and outer cams 9. The cylindrical projection 33 for a latch connection is applied at the outside and is perforated by passages 17 for better sterilisation.

A further example is shown in FIG. 7 in which the trial ball for a prosthesis ball is used whose conical counter surface 4 ends in a neck projecting over the ball surface. The insert 2 likewise has a beaker shape and is divided into flexion springs 5 by elongate slots 10. The flexion springs 5 have inner cams 6 which project over the conical counter surface 4 and which produce a holding force when pushed onto a cone 3. The flexion springs 5 are prevented from a further radial deflection by a support 35 at the trial ball 1 which engages in each case roughly at the half of the flexion springs 5 in order to achieve a centration at the height of the inner cams in addition to the centration in the beaker base.

1. Trial balls for hip joint prostheses which have a cone connection between a prosthesis neck (24) with a cone (3) and fittable hip joint balls, with the hip joint balls and the associated trial balls (1) each having practically the same radii (21) and each having the same position of their conical counter surfaces (4) relative to the ball centre (11), characterised in that the trial balls (1) have a separate insert (2) with a pre-determined spring effect for the contact to the cone (3), with the insert being able to be secured in the trial balls (1) at a different spacing (27) along the polar axis (13) to the ball centre (11).

2. Trial balls in accordance with claim 1, characterised in that trial balls (1) of one cone size have a same separate insert (2).

3. Trial balls in accordance with any one of the claims 1 or 2, characterised in that the trial balls (1) and the insert (2) are made of plastic.

4. A trial ball in accordance with any one of the claims 1 or 2, characterised in that the insert can be made as an injection moulding by an injection moulding tool.

5. A trial ball in accordance with claim 1, characterised in that the insert (2) is made of metal.

6. A trial ball in accordance with any one of the preceding claims, characterised in that the insert (2) has the shape of a beaker whose rim is divided by elongate slots (10) into support tongues (22) continuing the conical counter surface (4) and into flexion springs (5) with inner cams (6) projecting over the cone shape in order to guide the cone (3) with the support tongues (22) and to prevent sliding off by friction by the radial forces produced by the flexion springs (5).

7. A trial ball in accordance with claim 6, characterised in that the support tongues (22) are radially supported in the trial ball (1).

8. A trial ball in accordance with claim 6 or claim 7, characterised in that the flexion springs (5) of the insert (2) are provided with outer cams (9) which engage into elongate grooves (19) at the trial ball (1) for the securing of the insert (2) against rotation.

9. A trial ball in accordance with any one of claims 1 to 8, characterised in that the insert (2) is held in the trial ball (1) via a catch connection (26).

10. A trial ball in accordance with any one of claims 6 to 9, characterised in that the inner cams (6) on a rasp with a cone (3) engage into a channel (15) or into a cut-out (14) to prevent the trial ball (1) from sliding out.

11. A trial ball in accordance with any one of claims 1 to 5, characterised in that the insert (2) has the form of a beaker whose rim is divided by elongate slots (10) into flexion springs (5) with inner cams (6) projecting over the cone shape in order to produce a holding force via a deflection of the flexion springs (5) corresponding to the projection of the inner cams (6), with the flexion springs (5) being prevented from continuing the deflection by an additional support in the trial ball (1) in order to achieve a centration on the mounting onto a cone (3).

12. A trial ball in accordance with any one of claims 1 to 11, characterised in that the contact surfaces between the insert (2) and the trial ball (1) have passages (17) outwardly in order also to ensure sterilisation in the contact surfaces.
